(19) United States
(12) Patent Application Publication
(10) Pub. No.: US 2018/0000419 A1
(43) Pub. Date: Jan. 4, 2018

(54) AUTOMATED MEASUREMENT AND CONTROL SYSTEM FOR TATTOO DELIVERY

(71) Applicant: William Rassman, Los Angeles, CA (US)

(72) Inventor: William Rassman, Los Angeles, CA (US)

(21) Appl. No.: 15/541,248

(22) PCT Filed: Dec. 30, 2015
(86) PCT No.: PCT/US15/68187

§ 371 Date: Jun. 30, 2017

Related U.S. Application Data
(60) Provisional application No. 62/166,266, filed on May 26, 2015.
(60) Provisional application No. 62/097,926, filed on Dec. 30, 2014.

(51) Int. Cl. A61B 5/00; A61B 5/01; A61B 5/107; A61M 37/00

(52) U.S. Cl. CPC A61B 5/6848; A61M 37/0076; A61B 5/01; A61B 5/1079; A61M 2205/33

(57) ABSTRACT

Tattooing, or delivery of pigment into the skin, producing temporary or permanent markings on the skin, is practiced for aesthetic and practical purposes. Human and mechanical control of tattooing may be augmented by sensors, coupled to an adaptive control system capable of improving the results, and relieving demands on the operator.

### CROSS REFERENCE TO RELATED APPLICATIONS

This is a national phase application of International Application No. PCT/US15/68187, filed Dec. 30, 2015 which claims priority to U.S. provisional application No. 62/097,926 dated Dec. 30, 2014 and U.S. provisional application No. 62/166,266 dated May 26, 2015 the contents of which are hereby incorporated by reference.

### TECHNICAL FIELD

This invention relates to the delivery of tattoo ink to produce temporary or permanent markings on the scalp to mimic the appearance of a closely cut hair follicle. More specifically, this invention relates to an apparatus and method for tattooing capable of monitoring and controlling the depth and the quantity of the ink delivered by the tattoo device.

### BACKGROUND

In general, a tattooing apparatus is an apparatus that has traditionally been used to place a pigment underneath the skin surface to create signs, letters, a pattern or picture. Traditionally, the tattoo process uses variable needle designs, manual or motor control of the needle, and a high degree of operator input. Tattoos on the scalp to mimic single hair follicles are a subset of the traditional tattoo process with its own idiosyncrasies thus requiring special techniques and instrumentation.

The use of tattoos on the human scalp for camouflaging hair loss or scarring of the scalp by means of a stippling pattern, simulating the appearance of hair follicles or short hairs, is relatively new, and is termed Scalp Micro Pigmentation (SMP). SMP is at present performed by a micropigment instrument that can accommodate between 1-6 needles, which can cycle between 100-200 cycles per second. The operator uses the instrument to create individual marks on the scalp thus creating a ‘stippled’ effect over the entire scalp to resemble short or shaved hair follicles. At present, the operator is responsible for maintaining three dynamic variables: (1) The correct angle of the hand piece thus controlling the angle of needle entry against the scalp. (2) The pressure exerted on the hand piece thus controlling the depth of penetration of the needle into the scalp. (3) The time interval of the hand piece held on a single area, thus controlling the time interval that the needle is in contact with the scalp. This variable of time is affected by the cycling speed of the needle. In other words, the aforementioned time interval can also be seen as the number of times the needle is in contact with the scalp. At present, the cycle speed is not a dynamic variable as it is only adjusted once at the beginning of the procedure in most cases. The entire SMP process requires great attention, concentration, and repetitive use of both fine and gross motor skills with precise timing, especially recognizing that the scalp is not a homogenous canvas.

The human scalp is made up of two basic layers that is of interest for the application of SMP. The first and most superficial layer is the epidermis, which can vary in approximate thickness between 0.5 mm to 1.5 mm. Pigment or ink deposited in the epidermal layer is not considered permanent. The needles must pierce through the epidermis on its way to the second layer of the scalp, the dermis, where the pigment or ink is to be deposited for the permanence of the SMP. The dermal layer is not homogenous and its boundaries are relatively thick. Taking this variation into account, the outcome of SMP is dependent on depositing a minute amount of pigment or ink in a certain location (depth) of the dermal layer of skin. The goal of SMP is to deposit the pigment or ink in the upper section of the dermis just beneath the epidermal layer. As the layers of skin are not consistent on the same individual and the operator is constantly adjusting external control variables such as angle, pressure, and timing of the instrument as the uniform application of SMP over the entire scalp is challenging.

For convenience of tattoo procedures, a conventional tattooing apparatus is configured so that the tattoo needle(s) reciprocates at an adjustable speed or cycles per second, usually in the range of 100 cycles per second. The actual depth and time of the needle's penetration of the dermis is in practice controlled by the operator, pushing the tip onto the skin surface and manually ‘feeling’ the resistance encountered. Thereby the depth and number of needle strokes discharging ink varies according to the operator's application of force for a period of time exerted on the instrument over a location on the skin

When delivering a tattoo, the operator of the instrument is limited to two methods: a “feel” during actual placement of the tattoo ink, and visual feedback in assessing the result of tattooing. This limited set of parameters is actually dependent on a long list of factors:
Angle of needle(s) with respect to the normal surface of the skin (α)
Number of Strokes (Rate·Time or n)
Needle Diameter (0.20 mm-0.4 mm, microns)
Needle Number (1-9)
Needle Pattern (linear, radial, or a desired pattern)
Needle Sharpness (scale of 1 to 5)
Dwell (time μseconds or radians)
Depth (d_{needle}, 0.5-3.0 mm)
Force or Pressure (F, Newtons)
Ink Viscosity (η, (N·s)/m2)
Ink Particle Size (d, median diameter, nanometers)
Ink Spectral Response (λ, reflection spectrum)
Ink concentration (Cᵢ, ink particles per volume)
Hair Color (RGB coordinates)
Skin Tone (Δ_{s,} transmission and reflection function spectrum)
Epidermal Toughness (force to pierce with a standard point, Newtons kg·m·s⁻²)
Dermal-Subdermal Modulus (E, (Pa or N/m² or m⁻¹·kg·s⁻²))
Dermal-Subdermal Thickness (t, mm from epidermal-dermal junction to bottom of fat.)
Local temperature of skin (T)
Local vascularity of skin (V)

Control and consistency of depth is critically important. To produce a stable tattoo, with pigment residing within the upper dermis at the dermal/epidermis boundary, the needles must place a controlled amount of the tattoo pigment just below the epidermis. The overall distance between the skin and the skull, and the relative depth of each layer varies between individuals, based upon age, genetics, weight, the health of the scalp, the amount of hair in the scalp, and the presence of scars in the scalp. The biomechanical/viscoelastic behavior of skin varies greatly from individual to individual, and from one region to another in the scalp on the same individual. Furthermore, every small region of the scalp has local variations in the contour of the underlying gross anatomy of the scalp, with irregular webs of connective tissue, pockets of fat, structures such as follicles and glands, underlying vasculature, miscellaneous scars and granulations, including those from previous surgeries or injuries, that microscopically change the anatomy of the scalp on a scale of millimeters.

The operator of the tattooing instrument learns, after long experience, to maintain a constant feedback loop by checking their progress, and making necessary adjustments to their technique and equipment settings continually along the way. The operator develops a ‘feel’ of the anatomy and mechanical properties of the skin on each patient at each point on the patient's scalp, however, this is recognized as physically and mentally exhausting and failure-prone. Furthermore, the operator cannot proactively address the state, location and quantity of the pigment spot by spot as it is being delivered without stopping to clean and examine the surrounding area, severely reducing the rate at which they work. Consistency and continuity of results may be lost if the operator pauses, checks their work, gets distracted or otherwise interrupted, or becomes fatigued. Consistency and continuity also suffers when different operators are working on the same patient. If the operator should alternate with another operator, the knowledge is not easily transferred and thus consistency is also affected. The results therefore, of one operator may be different when performed by another operator on the same patient. It is impractical to expect an operator to deliver the ideal depth and hold that depth for an ideal time to deliver the ideal amount of pigment. As the depth of the epidermis varies on an individual, and from one region of the scalp to another, and on a continually changing microscopic local scale, simply setting a depth gauge to control the depth is ineffective. Similarly, setting a single value for the dwell time in the skin, or the number of strokes is similarly ineffective. Furthermore, even a trained operator cannot maintain this effort, and repeat an exacting task to possibly over 50,000 spots on a single patient's scalp. In other words, even a trained operator cannot maintain a consistent SMP pattern over the entire scalp where thousands of spots are to be imprinted

It has been observed that leaving the needle in the epidermis for too many cycles produces an enlarged and over-traumatized wound, which leaks ink from the surface. If the instrument is held in the upper dermis for too long a period of time, delivering too many needle strokes, or the needle pushed deeper into the dermis, the ink could spread both deeper into the dermis and laterally causing a diffuse amalgam of ink and surrounding tissue to appear, rather than a discrete spot of ink. The diffuse amalgam of ink is not a successful stippled result for SMP. These events happen in milliseconds, and fine conscious control is not possible, thus the use of sensors and automated controls is the best means to control the variables discussed above.

One aspect of tattooing single discrete areas or spots on the scalp recognized by the inventor is that there is a change in the ‘feel’ during the delivery of each spot. There is an initial resistance from the hand-piece as it contacts the skin surface, but then as it penetrates the epidermis, the vibrations felt by the operator as the needle works its way into and through the epidermis to the upper fatty dermis, decreases. This is hypothesized to be because the needles penetrate a spot in the tough, hide-like epidermis repeatedly, and with the advance of the needle into and through the epidermis the resistance is due to the initial breaking of structures within the epidermis, the widening of the initial hole with each subsequent cycle of the instrument, and as the wound is wetted by the ink delivered to the epidermis. A significant reduction of resistance is noted once the needle(s) pass through the epidermis and enters the upper dermis.

It is intuitively obvious that if needles are simply wet, the friction between the needle and the tattooing head will be less than when dry. This will at once reduce resistance to motor movement, allowing an increase in the rate and reducing electrical resistance and thus the current load, all of which can be detected electrically and acoustically.

Using the control schema proposed, ideal depth and additional or fewer strokes may be achieved and consistently delivered just as needed, without imposing any further demands on the operators' attention. Currently, the needle depth is controlled by human touch and feel. Needle cycle duration is fixed although the rate and duration can be adjusted the adjustment is not dynamic. The angle of needle penetration is operator dependent and usually between sixty to ninety degrees (60-90°). Temperature control over a local area is something that may affect how the pigment goes in as well as lessen the pain. Cool skin has lower blood flow and keeps pigment from bleeding out. Currently, the number of needles is fixed at three as being optimal but it would be an advancement in the art if the number of needles could vary this dynamically, for example switching between one to three needles. Currently, it is common to use antibacterial based petroleum on the skin but it is possible to add adjuvants to the ink to promote healing of the skin, for example a steroid.

### NON-PATENT CITATIONS

A A Mehryar Mohri, Afshin Rostamizadeh, Ameet Talwalkar (2012) Foundations of Machine Learning, The MIT Press ISBN 9780262018258.

Cua et al. “Elastic Properties of Human Skin: Relation to Age, Sex and Anatomical Region” Archive of Dermatological Research, August 1990, Volume 282, Issue 5, pp 283-288.

Small et al. “Effect of dermal thickness, tissue composition, and body site on skin biomechanical properties” Skin Research and Technology, Volume 12 No. 1 pp 43-49

Hendriks “Mechanical behaviour of human epidermal and dermal layers in vivo” Doctoral Thesis, Technical University of Eindhoven March 2005.

### PATENT CITATIONS

U.S. Pat. No. 8,228,666 issued to Rickard titled Retrofit control system and power supply for a tattoo gun discloses a compact, wearable, battery-powered supply and controller.

U.S. Pat. No. 8,171,825 issued to Adams titled magnetic coil tattooing machine discloses an improved actuator/interrupter switch, removing spring tuning.

U.S. Pat. No. 8,091,585 issued to Cooper titled Pneumatic regulator unit and method of use discloses a manifold and valves for operating multiple tattoo heads from a single pressure source.

U.S. Pat. No. 7,969,715 issued to Copeland titled Power supply for a tattoo machine discloses fundamentally a patent for a power supply housing amenable to cleaning.

U.S. Pat. No. 7,748,294 issued to Garitano titled Device for tattooing and method of using the same discloses single coil and PWM to reduce weight, eliminate set screw depth adjustments.

U.S. Pat. No. 7,695,486 issued to Dixon titled Intradermal color introducing needle device, and apparatus and method involving the same discloses good references to the art; arrangements of a plurality of needles at depths best to address eyelids and eyebrows, conventional drive means and control.

U.S. Pat. No. 6,689,095 issued to Garitano titled Needleless permanent makeup and tattoo device discloses entirely different method of ink delivery without respect to sensing and control.

U.S. Pat. No. 6,685,719 issued to Matera titled Surgical tattooing apparatus and method discloses single-use reservoir of sterile ink for marking radiation medicine patients.

U.S. Pat. No. 6,550,356 issued to Underwood titled Tattoo technology discloses cordless tattoo needle bar power head with wireless (foot) on-off switching.

U.S. Pat. No. 6,282,987 issued to Moniz titled Contact bar assembly for a tattooing device discloses Minor mechanical improvement to standard tattoo needle bar power head.

U.S. Pat. No. 5,471,102 issued to Becker titled Reciprocating shaft device discloses a rationalized lightweight hand piece, linear drive and square wave drive pulse scheme.

U.S. Pat. No. 5,401,242 issued to Yacowitz titled Apparatus for injecting a substance into the skin discloses continues previous, adds depth control via annular ring and foot.

U.S. Pat. No. 5,054,339 issued to Yacowitz titled Tattooing assembly discloses an electric pump for delivering ink to the needles.

U.S. Pat. No. 4,914,988 issued to Chang titled Eyebrow tattooing machine discloses miniaturized and battery-powered, otherwise conventional motor and cam drive.

U.S. Pat. No. 4,508,106 issued to Angres titled Microsurgical method for applying permanent eyelid liner discloses surgical steps for eyelid tattoos, no relevant drive control matter.

### SUMMARY OF THE INVENTION

The exemplary embodiment of the present invention addresses the depth control problem, along with control of the volume of the pigment that is placed into the upper dermis within 2 mm of the basement layer of the epidermis. Accordingly, an aspect of the present invention is to provide a tattooing apparatus, which can sense and then control the depth at which the apparatus operates and the number of strokes that discharge pigment.

This invention permits the operator to teach the apparatus, and thereafter simply “point and shoot,” bringing the needle to the skin with a fixed force applied with the operator's hand, and leaving it in position until the needle is halted automatically. The operator's hand as applied to the tattoo apparatus presently controls force, depth, and number of strokes to achieve a consistent spot. No monitoring of “feel” is required with this invention and no rapid “jerk” withdrawal is necessary achieve best results for the tattoo recipient. The control of the cycle permits the needles to be left in the withdrawn position thereby sparing additional piercings of the skin, and allows the operator to move to the next location by removing the instrument from the patient's skin and advancing it to the next location. An important aspect of the invention is to reduce the exposure to occupational injury. Tattoo artists, in particular those specializing in “stippled” patterns like Scalp Micro-Pigmentation (“SMP”) are known to incur repetitive stress disorders such as carpal tunnel syndrome and the subject invention may reduce the wrist and arm movements contributing to such injuries. Similarly, halting the needle movement upon completion of a spot has an additional benefit of minimizing the duty cycle of operation, directly reducing the operator's time of exposure to the 50-150 Hz vibrations most commonly cited as the cause of hand-arm-vibration syndrome (HAVS), and providing destructive mechanical or acoustic interference may also further reduce vibration exposure.

A further aspect of the present invention is to provide a tattooing apparatus which can be easily used by a person with less skill in the stippled tattooing process. Furthermore the wetting behavior of different substances, such as water vs saline vs ink vs blood vs viscous or encrusting clot material comprises a gradient of resistance and acoustic damping that allows identification of what is wetting the needle. This has the primary advantages of permitting control of inking of the needles and addressing cleanliness of the tip. Thus the operator's attention to the amount of ink may be reliably relieved, and the situation where the needle has become fouled by accumulated material may be detected readily.

A further aspect of the present invention is to provide for depositing a predetermined amount of tattooing ink into the fatty subdermal layer of the scalp with a device, said device comprising a depth sensor responsive to (i) a change in needle feedback signal as the needle interacts with the skin (this way it can be interpreted as before or after the needle enters the skin) and (ii) a change in needle position when viewed by a sensor (the sensor can be ultrasound, light, heat, camera, voltage, or amperage drawn by the electrical elements of the sensor).

To control the (a) needle depth; (b) cycle speed of needle; (c) cycle duration of needle; (d) angle of penetration of needle; (e) temperature of local working area and an ink to be deposited for curing and stabilizing purpose; (f) number of needles penetrating the skin; (g) additional delivery of stabilizing healing compound and a sensor with a microprocessor that controls the optimal delivery of ink in the skin for a desire effect.

### BRIEF DESCRIPTION OF THE DRAWINGS

FIG. 1 shows a schematic of a traditional tattoo apparatus including a simple electrical power source and handpiece.

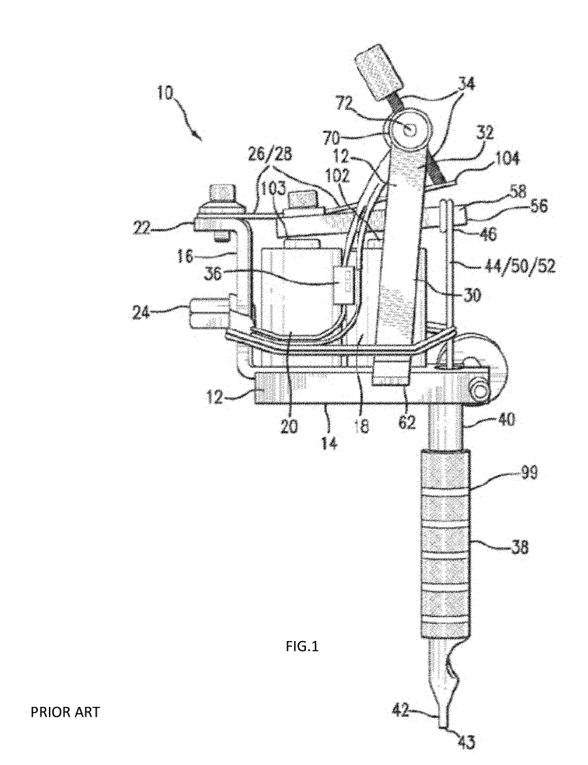

FIG. 2 shows a photograph of a satisfactory dense and consistent scalp pigmentation result.

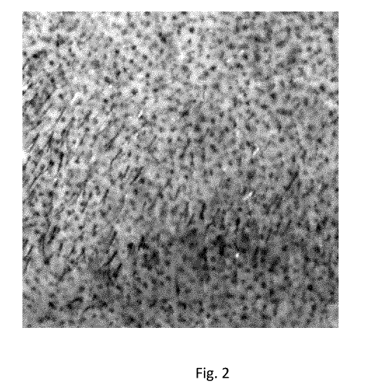

FIG. 3 shows a photograph of an unsatisfactory result including inconsistent size and density of stippling.

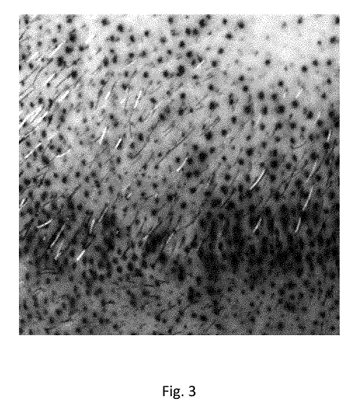

FIG. 4 shows a histology image of pigment particles in and among the fibroblasts at the dermis/epidermis border.

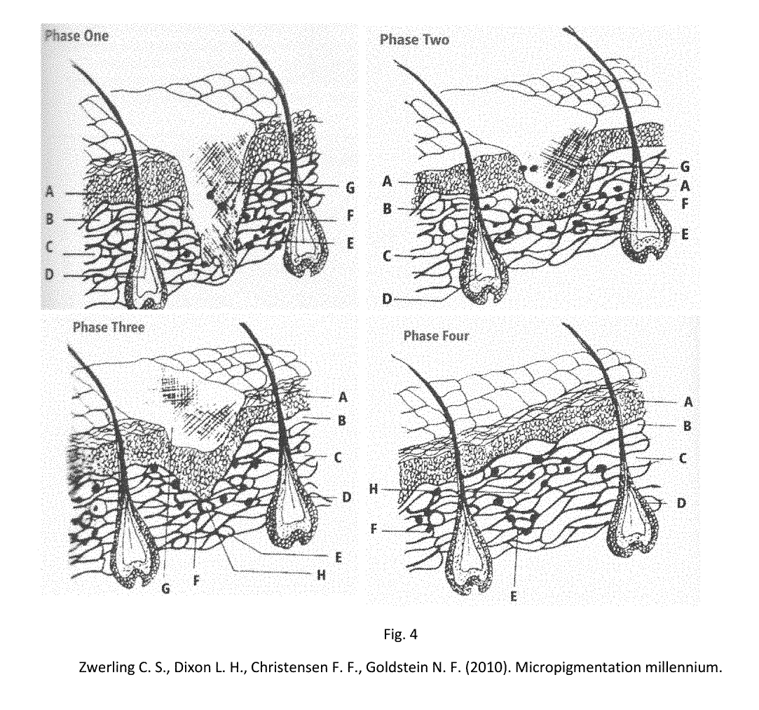

FIG. 5 shows a histology image of pigment particles diffused into the dermis.

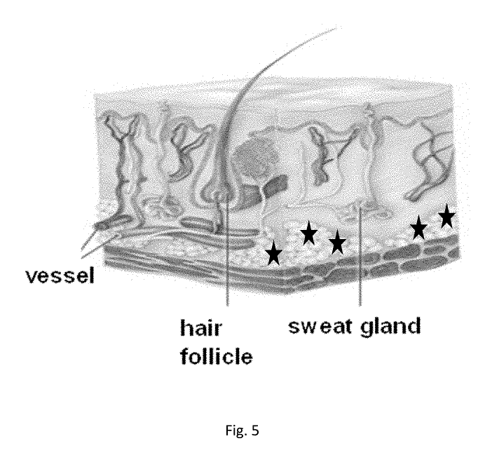

FIG. 6 shows a schema for control, consisting of the four elements of the apparatus: the anatomy; the tattooing device, the electro-acoustic sensors and transducers; and the processing unit.

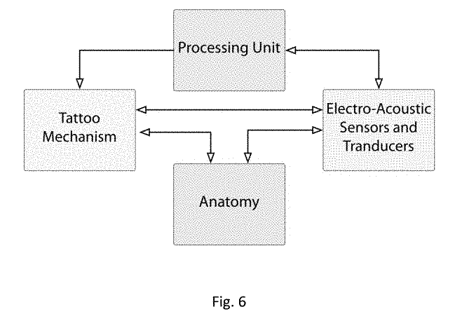

FIG. 7 shows an illustration of the phases of the method.

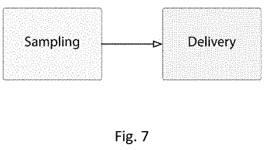

### DETAILED DESCRIPTION

According to one aspect of an exemplary embodiment of the present invention, there is provided a tattooing apparatus, including tattoo needle(s) cycling back and forth into the skin to allow a pigment solution to penetrate beyond the epidermis and into the upper dermis for a given period of time and at a controlled depth. While referred to herein as a pigment solution it is understood that there are numerous commercially available inks and pigments for tattooing. It should also be understood that an inventive aspect of the invention is the incorporation of additional adjuvants such as collagen or collagen production stimulating chemicals, proteins or other naturally occurring collagen stimulants. Pigment solutions could have additional clinically beneficial levels of vitamins.

According to another aspect of an exemplary embodiment of the present invention, there is provided a tattooing apparatus, including a main body with a handle portion having a mechanical drive such as a motor therein and a series of needle(s) cycling for discharging tattoo pigment.

The tattooing apparatus may further include mechanical, electronic and acoustic sensing elements which can detect changes in the state of operation of the needle as a result of mechanical resistance from the cycling of the tattoo needle through the epidermis and into the upper dermis from among the following inputs: signals from an encoder for determining direction of rotation and position and a zero or index or reference signal encoder that counts revolutions/frequency, resistance, amperage, voltage, back-EMF velocity, radio-frequency emissions, and acoustic emissions. These encoder inputs work in cooperation to allow the needle to operate as having multiple sensors, primarily for determining relative resistance of needle movement as it relates to biomechanical properties of the skin. The encoder could utilize position sensing devices like gyros or accelerometers to help orient an operator.

The needle of the tattooing apparatus thus provided serve as a primary sensor for contact with the epidermis, the viscoelastic properties of the dermis, the depth of the dermis, and the degree to which skin has been repeatedly punctured when delivering ink. Each stroke of the needle as it goes through a cycle encountering different conditions produces a characteristic curve of mechanical, electrical and sonic feedback, and the frequency will be variably retarded by contact with materials of differing density and with different fractional drag on the needle, which may be monitored and analyzed by the encoders. Motors can be alternating current, direct current, coil, other commonly commercially available in today's tattoo devices.

In an alternative embodiment the device can be operated by a robot in lieu of a human operator. Robot operation is possible via communication between a microprocessor of a preferred embodiment with a robot capable of movement in three-axis about a patient scalp. In this alternative embodiment the operator could be a smart robot that could be positioned about a patients head. In this robot embodiment the device could scan the patients head and determine the proper orientation for delivering ink based on the previously described factors.

A processing unit is a computer, or a microprocessor such as Field Programmable Gate Arrays (“FPGA”), equipped with a user interface and algorithms capable of learning the ideal technique for each individual patient's anatomy from a human operator, and the processing unit is capable of adjusting the operation of the tattoo apparatus, including mechanical adjustments and delivery of power from the main body unit so as to maintain the needle depth at the proper position during any point in a cycle. Commercially available Xilinx Field Programmable Gate Arrays (FPGAs), holds multiple patents, and is the clear market leader in programmable logic in terms of both revenue and technology.

In a preferred embodiment a method consists of two phases, wherein a user first teaches a tattoo apparatus, and a second wherein the tattoo apparatus adaptively adjusts operational parameters to achieve the result desired consistently for the duration of the procedure based on the learning from the first phase. For example, during a first sampling phase an operator applies a small statistically significant sample, perhaps ten sites or spots, over a limited area, utilizing the operator's manual control of all parameters and a visual subjective judgment of success, while the tattoo apparatus encoders are collecting data on each cycle. The processing unit analyzes the inputs that include most significantly, depth of penetration of a needle, number of strokes at a correct depth, and withdrawal of the tattoo needle from the skin at the precise moment that the spot is optimally positioned. These inputs may then be processed by means of supervised learning algorithms well-established in the field of machine learning. This establishes and teaches the tattoo apparatus a norm or mean condition for the patient's skin as well as the necessary adaptive responses to conditions outside the norm or mean condition. During the delivery phase the tattoo apparatus collects data from the encoders during delivery of each spot, and using what the processing unit has learned, controls speed and/or force and depth of the needle stroke and the frequency of strokes to deliver the optimum amount of pigment solution at the optimum position. Also, the number and operation of the needles may operate independently. Furthermore there may be a vibrating element such as a piezoelectric crystal or small motor to serve as a second acoustic ping generator for monitoring a needle state, with similar drive sensing capabilities, and secondly to provide vibrations to generate destructive interference for the primary mechanical vibration.

Thus after the first phase, the first few strokes in a subsequent scalp location are used to sense and analyze, and the remaining needle strokes are adjusted to best address local scalp conditions. These factors in tandem with the operator's input during the first phase permit control of tattooing of each spot. For instance, if a spot has an area of thickened epidermis, the needle will encounter greater resistance than normal early in the stroke, just as the needle contacts and pierces the epidermis, detectable in the amplitude and/or period, followed by normal completion of the duration of the pierce/withdraw cycle. Upon sensing this changed condition, the processing unit can fit to the curve to determine the nature of the differing condition, and calculate the area of the difference or perturbance of the “normal” cycle, and adjust accordingly. Simple mechanical adjustments such as a cam follower or adjustable depth plate to deepen or change the duration of different phases of the stroke are well known in the mechanical engineering art, though not present in the field. In a preferred embodiment, additional electrical power may be supplied to the motor or the waveform of electrical power delivery may be adjusted, increasing the velocity of the needle to compensate for tougher anatomy. It should be understood that a needle cycle may also encompass a vibration motion that is not of a strictly vertical trajectory relative to the epidermis, for example side to side, circular or other patterns in a plane or with slight changes in vertical penetration and retraction of the needle relative to the skin surface.

The encoders described herein are not intended to be limited to the numerous commercially available types of sensors commercially available but are disclosed to aid in the enablement of the tattoo apparatus. Temperature sensors come in the form of thermocouples, resistance-temperature detectors, and thermistors. Thermistors differ from resistance temperature detectors (“RTDs”) in that the material used in a thermistor is generally a ceramic or polymer, while RTDs use pure metals. The temperature response is also different; RTDs are useful over larger temperature ranges, while thermistors typically achieve a higher precision within a limited temperature range, typically −90° C. to 130° C. Infrared sensors emit and/or detect infrared radiation to sense a particular phase in the environment. The infrared sensor may detect radiation differences between dermis with and without pigment embedded which is not visible to human eye. Similarly, ultraviolet (“UV”) sensors measure the intensity or power of the incident ultraviolet radiation. This form of electromagnetic radiation has wavelengths longer than x-rays but is still shorter than visible radiation. An active material known as polycrystalline diamond is being used for reliable ultraviolet sensing. UV sensors can measure how much pigment has been delivered to the dermis.

In another preferred embodiment ultrasonic or light sensitive transducers can measure thickness and density of the epidermis and differentiate it from the sub-dermal fat immediately below the epidermis. The needles themselves, will be made of a solid material which will easily be detected in relationship to the surrounding anatomy of all layers of the skin. These transducers can measure these solid needles as they move into and through the epidermis to the exact point at which the tattoo needles enter the sub-dermal space. As the pigment has a higher density than the fat in the sub-dermal space, the pigment can be visualized as it builds into an aggregate of some dimension. As the pigment is deposited in the sub-dermal space immediately below the epidermis, the pigment can be seen with ultrasonic or optical sensors as it accumulates in the sub-dermal space. Ultrasonic and/or optical sensors can measure the physical dimensions of the pigment aggregate deposited in the sub-dermal space because of these density differences between the fat (which has density close to water) and the pigment amalgam which will have a higher density than water and may contains minutes amounts of metallic molecules which will reflect both sound and/or light. By measuring both the point at which the needles penetrate the epidermis and the size of the pigment aggregate, precise feedback signals can be obtained to stop pigment deposition once the ideal depth and the aggregate size has been determined.

Today's pigments include the original mineral pigments, modern industrial organic pigments, a few vegetable-based pigments, and some plastic-based pigments. Allergic reactions, scarring, phototoxic reactions (i.e., reaction from exposure to light, especially sunlight), and other adverse effects are possible with many pigments.

Plastic-based pigments are very intensely colored, but many people have reported reactions to them. There are several pigments that glow in response to black (ultraviolet) light. These pigments are notoriously risky—some may be safe, but others are radioactive or otherwise toxic. The oldest natural pigments come from ground up minerals and carbon black (one of the main constituents of Indian Ink). Some of the minerals most commonly used as natural pigments are Mica (AKA pearlescent powder) Mica dust, and pearl pigments, both are cosmetic grade, and also approved by the FDA. Natural colorants are ideal cosmetic pigments they are water dispersible and about one teaspoon of colorant will easily color four pounds of soap. A wide range of bismuth oxychloride and liquid food coloring are used widely as cosmetic pigments.

Unnatural color pigments however yield a more intense color than natural cosmetic pigments and are water dispersible, transparent and blend well with soap. Pigment blends include yellow pigment blend made of titanium dioxide with FDA approved iron oxide, iron oxide brown and iron oxide red and red based pigment blend which contains titanium dioxide with FDA approved, iron ocher, iron oxide brown and iron oxide red.

In addition to the pigment Tattoo ink consists of a carrier. The carrier may be a single substance or a mixture. The purpose of the carrier is to keep the pigment evenly distributed in a fluid matrix, to inhibit the growth of pathogens, to prevent clumping of pigment, and to aid in application to the skin. Among the safest and most common ingredients used to make the liquid are:
ethyl alcohol (ethanol)
purified water
witch hazel
Listerine
propylene glycol
glycerine (glycerol)

A key consideration is that cosmetic pigments might oxidize and fade over a period of time and might require a touch ups. The application of pigment should always include a thorough knowledge of the shape selection, selection of techniques, pain and swelling control and conservative applications.

The table below lists colors of common pigments use in tattoo inks. It should be noted that many inks mix one or more pigment¹: ¹http://chemistry.about.com/library/weekly/aa121602a.htm

Composition of Tattoo Pigments

| Color | Materials | Comment |
|---|---|---|
| Black | Iron Oxide (Fe3O4) | Natural black pigment is made from magnetite crystals, powdered jet, |
| | Iron Oxide (FeO) | wustite, bone black, and amorphous carbon from combustion (soot). |
| | Carbon | Black pigment is commonly made into India ink. |
| | Logwood | Logwood is a heartwood extract from Haematoxylon campechisnum, |
| | | found in Central America and the West Indies. |
| Brown | Ochre | Ochre is composed of iron (ferric) oxides mixed with clay. Raw ochre is |
| | | yellowish. When dehydrated through heating, ochre changes to a reddish |
| | | color. |
| Red | Cinnabar (HgS) | Iron oxide is also known as common rust. Cinnabar and cadmium |
| | Cadmium Red (CdSe) | pigments are highly toxic. Napthol reds are synthesized from Naptha. |
| | Iron Oxide (Fe2O3) | Fewer reactions have been reported with naphthol red than the other |
| | Napthol-AS pigment | pigments, but all reds carry risks of allergic or other reactions. |
| Orange | disazodiarylide and/or | The organics are formed from the condensation of 2 monoazo pigment |
| | disazopyrazolone | molecules. They are large molecules with good thermal stability and |
| | cadmium seleno-sulfide | colorfastness. |
| Flesh | Ochres (iron oxides mixed | |
| | with clay) | |
| Yellow | Cadmium Yellow (CdS, | Curcuma is derived from plants of the ginger family; aka tumeric or |
| | CdZnS) | curcurmin. Reactions are commonly associated with yellow pigments, in |
| | Ochres | part because more pigment is needed to achieve a bright color. |
| | Curcuma Yellow | |
| | Chrome Yellow (PbCrO4, | |
| | often mixed with PbS) | |
| | disazodiarylide | |
| Green | Chromium Oxide (Cr2O3), | The greens often include admixtures, such as potassium ferrocyanide |
| | called Casalis Green or | (yellow or red) and ferric ferrocyanide (Prussian Blue) |
| | Anadomis Green | |
| | Malachite [Cu2(CO3)(OH)2] | |
| | Ferrocyanides and | |
| | Ferricyanides | |
| | Lead chromate | |
| | Monoazo pigment | |
| | Cu/Al phthalocyanine | |
| | Cu phthalocyanine | |
| Blue | Azure Blue | Blue pigments from minerals include copper (II) carbonate (azurite), |
| | Cobalt Blue | sodium aluminum silicate (lapis lazuli), calcium copper silicate (Egyptian |
| | Cu-phthalocyanine | Blue), other cobalt aluminum oxides and chromium oxides. The safest |
| | | blues and greens are copper salts, such as copper pthalocyanine. Copper |
| | | pthalocyanine pigments have FDA approval for use in infant furniture and |
| | | toys and contact lenses. The copper-based pigments are considerably |
| | | safer or more stable than cobalt or ultramarine pigments. |
| Violet | Manganese Violet | Some of the purples, especially the bright magentas, are photoreactive |
| | (manganese ammonium | and lose their color after prolonged exposure to light. Dioxazine and |
| | pyrophosphate) | carbazole result in the most stable purple pigments. |
| | Various aluminum salts | |
| | Quinacridone | |
| | Dioxazine/carbazole | |
| White | Lead White (Lead | |
| | Carbonate) | |
| | Titanium dioxide (TiO2) | |
| | Barium Sulfate (BaSO4) | |
| | Zinc Oxide | |

Various pigments have differing amounts of metallic pigments and it is understood that ultrasonic and/or optical pigment characteristics can be added, reduced or removed to optimize the sensitivity of the detector means. ¹http://chemistry.about.com/library/weekly/aa121602a.htm

Electrical sensors are inexpensive, accurate, and readily available in many designs. Electrical sensors can be directed towards current, voltage, or power.

Piezoelectric pressure sensors work under rapidly changing conditions and would be a preferable embodiment.

Ultrasonic transducers can measure thickness and density of skin.

Polarized and depolarized light can be used to visualize blood vessels and the anatomy of the skin.

1. A tattooing device comprising one or more needles operative for moving pigment from a reservoir to the needle tip(s) for delivering pigment to the upper dermal layer of a selected location in the scalp of a patient, said device comprising one or more sensors for dynamically sensing changes in skin characteristics as said needle(s) penetrates the skin of the scalp, said sensor(s) being operative to signal the arrival of the needle tip(s) at the upper dermal layer of the selected location in the upper dermis.

2. A device as in claim 1 wherein said sensors comprise one or more of an electrical, pressure, temperature, sound or optical signals.

3. A device as in claim 1 comprising one or a plurality of hollow or solid core needles.

4. A device as in claim 1 comprising a microprocessor operative responsive to said signal(s) to determine the depth of penetration of said needle or needles and the dwell time of said needle or needles.

5. A device as in claim 4 wherein said device is adapted for penetrating the skin at a 45 degree angle or at a 90 degree angle.

6. A device as in claim 4 wherein said microprocessor is operative to control said depth of penetration and dwell time to deliver a prespecified amount of pigment to the selected location of the scalp at a depth of the upper fatty upper dermis at the selected location.

7. A device as in claim 3 adapted for human or robotic manipulation.

8. A device as in claim 4 comprising one or a plurality of hollow or solid needles adapted for human or robotic manipulation.

9. The device as in claim 4 wherein said device further comprises an actuator, needle injector, or a variable speed motor and a rotating cam follower and a screw adjustment to the needle extension operative to extend said one or more needles in response to said first signal or microprocessor output.

10. The device of claims 1 wherein the needle depth into the skin is between 0.3 mm-4.0 mm,

11. The device of claims 1 wherein the needle dwell time into the skin is between 1.0 seconds-0.005 seconds.

12. A device as in claim 4 comprising a plurality of needle assemblies, each of said needle assemblies comprising one or more needles, said microprocessor being operative to control the penetration and dwell times of each of said assemblies.

13. The device of claims 1 wherein the microprocessor utilizes feedback from a feel from a human hand or sensed by a robot operator to predetermine a precise delivery of pigment based upon a learning algorithm.

14. The method of delivering a prescribed amount of material through the skin and into the upper fatty dermis through a tattoo instrument, said method comprising the steps of: 1. advancing the tattoo instrument of claim 1 into the skin at a selected location; 2. observing the sensor(s) for depth limiting signal; 3. activating the tattoo instrument for delivering pigment through the needle or needles in the tattoo instrument when the depth-limiting signal occurs

15. The method of delivering a prescribed amount of material through the skin and into the upper fatty dermis through a tattoo instrument, said method comprising the steps of: 1. advancing the tattoo instrument of claim 14 into the skin at a selected location; 2. observing the sensor(s) for feedback from pressure, electrical, temperature, sound or optical signals indicating a volume of pigment; 3. activating the tattoo instrument for delivering pigment through the needle or needles in the tattoo instrument when the volume signal occurs which can limit the volume delivered dynamically.

16. The method of delivering a prescribed amount of material through the skin and into the upper fatty dermis through a tattoo instrument of claim 15, said method comprising the steps of: 1. advancing the tattoo instrument of claim 1 into the skin at a selected location; 2. wherein the sensor(s) is processed by a microprocessor; 3. activating the tattoo instrument for delivering pigment through the needle or needles in the tattoo instrument wherein the microprocessor control and adjusts the dwell time and/or the depth of the needle into the upper fatty dermis dynamically.

17. The method of delivering a prescribed amount of material through the skin and into the upper fatty dermis through a tattoo instrument of claim 16, said method comprising the steps of: 1. advancing the tattoo instrument of claim 1 into the skin at a selected location; 2. wherein the sensor(s) signals are processed by a microprocessor; 3. activating the tattoo instrument for delivering pigment through the needle or needles in the tattoo instrument further comprising an actuator, needle injector, or a van able speed motor and a rotating cam follower and a screw adjustment to the needle extension operative to (a) extend said one or more needles cyclically in response to said first signal, and/or (b) dynamically adjust the needle injector or variable speed motor and/or a rotating cam followers and screw adjustment to the needle to the sensor signals in response to the microprocessor output.

18. The method of delivering a prescribed amount of material through the skin and into the upper fatty dermis through a tattoo instrument of claim 15, said method comprising the steps of: 1. advancing the tattoo instrument of claim 1 into the skin at a selected location; 2. wherein the sensor(s) is processed by a microprocessor; 3. activating the tattoo instrument for delivering pigment through the needle or needles in the tattoo instrument, 4, delivering a precise amount of pigment to a patient's skin of claim 17 wherein (a) said method dynamically adjust the needle depth between 0.5 mm-4.0 mm in response to sensing signals and microprocessor output

19. The method of delivering a precise amount of pigment to a patient's skin of claim 16 wherein said method dynamically (a) adjusts the needle dwell time between 1 seconds-0.005 seconds to (b) deliver a specified amount of pigment.

20. The method of delivering a precise amount of pigment to a patient's skin of claimn 16 wherein said method adjusts the needle to (a) 45 degrees, to (b) 90 degrees or (c) to some needle angle relative to the skin between 45-90 degrees and adjust the delivery in response to a pressure signal, an electrical signal, a temperature signal, a sound signal, an optical signal or the output of a microprocessor to change the delivery of the pigment into the skin.

21. The method of delivering a prescribed amount of material through the skin and into the upper fatty dermis through a tattoo instrument of claim 16, said method comprising the steps of: 1. advancing the tattoo instrument of claim 1 into the skin at a selected location; 2. wherein the sensor(s) is processed by a microprocessor; 3. activating the tattoo instrument for delivering pigment through the needle or needles in the tattoo instrument operated by, (a) a human hand or (b) a robot either responsive to a pressure signal, an electrical signal, a temperature signal, a sound signal, optical signal or to a microprocessor output to change the delivery of the pigment into the skin.

22. The method of delivering a prescribed amount of material through the skin and into the upper fatty dermis through a tattoo instrument of claim 15, said method comprising the steps of: 1. advancing the tattoo instrument of claim 14 into the skin at a selected location; 2. wherein the sensor(s) is processed by a microprocessor; 3. activating the tattoo instrument for delivering pigment through the needle or needles in the tattoo instrument, wherein the microprocessor utilizes feedback from (a) a feel from a human hand or (b) sensed by a robot operator to predetermine a precise delivery of pigment based upon or (c) a learning algorithm, further adjusted by input of a pressure signal, an electrical signal, a temperature signal, a sound signal, or an optical signal to change the delivery of the quantity of pigment into the upper fatty dermis.
